(19) United States
(12) Patent Application Publication
(10) Pub. No.: US 2025/0090033 A1
(43) Pub. Date: Mar. 20, 2025

(54) METHOD FOR END-TO-END CUFF-LESS BLOOD PRESSURE MONITORING USING ECG AND PPG SIGNALS

(71) Applicant: SAMSUNG ELECTRONICS CO., LTD., Suwon-si (KR)

(72) Inventors: Suhas BETTAPALLI NAGARAJ, State College, PA (US); Yashas Malur Saidutta, Menlo Park, CA (US); Rakshith Sharma Srinivasa, Sunnyvale, CA (US); Jaejin Cho, Mountain View, CA (US); Ching-Hua Lee, Mountain View, CA (US); Chouchang Yang, San Jose, CA (US); Yilin Shen, Mountain View, CA (US); Hongxia Jin, San Jose, CA (US)

(73) Assignee: SAMSUNG ELECTRONICS CO., LTD., Suwon-si (KR)

(21) Appl. No.: 18/883,902

(22) Filed: Sep. 12, 2024

Related U.S. Application Data
(60) Provisional application No. 63/539,546, filed on Sep. 20, 2023.

(51) Int. Cl. A61B 5/021; A61B 5/00; A61B 5/024; A61B 5/318

(52) U.S. Cl. CPC A61B 5/02125; A61B 5/318; A61B 5/681; A61B 5/02416

(57) ABSTRACT

A method for performing cuffless blood pressure (BP) measurement, including: obtaining a first physiological signal and a second physiological signal associated with a user; providing the first physiological signal as an input to a first transformer model; providing the second physiological signal as an input to a second transformer model; providing an output of the first transformer model and an output of the second transformer model as inputs to a third transformer model; providing an output of the third transformer model to at least one BP estimation model; and generating an estimated BP value corresponding to the first physiological signal and the second physiological signal based on an output of the at least one BP estimation model

### CROSS-REFERENCE TO RELATED APPLICATIONS

This application is based on and claims priority under 35 U.S.C. § 119 to U.S. Provisional Patent Application No. 63/539,546, filed on Sep. 20, 2023, in the U.S. Patent and Trademark Office, the disclosure of which is incorporated by reference herein in its entirety.

### BACKGROUND

### 1. Field

The disclosure relates to blood pressure (BP) monitoring, and more particularly to end-to-end cuffless BP monitoring using multimodal physiological signals.

### 2. Description of Related Art

Hypertension is a significant health concern and is a major contributor to cardiovascular diseases. As a result, blood pressure readings, including systolic blood pressure (SBP) values and diastolic blood pressure (DBP) values, are important cardiovascular health indicators.

Many approaches to blood pressure (BP) monitoring, such as oscillometric techniques and auscultatory techniques, rely on cuff-based devices to measure BP values. Although reliable, they are not suited for continuous monitoring and are not convenient to set up and use. These limitations have fueled research into more convenient, noninvasive methods, such as cuffless BP monitoring techniques.

Many cuffless BP monitoring techniques leverage electrocardiogram (ECG) and photoplethysmogram (PPG) signals, but are constrained by the need for handcrafted feature extraction. This may limit the generalizability of such models to diverse populations.

### SUMMARY

One or more embodiments of the disclosure are directed to end-to-end cuffless BP monitoring using multimodal physiological signals.

In accordance with an aspect of the disclosure, a method for performing cuffless blood pressure (BP) measurement includes: obtaining a first physiological signal and a second physiological signal associated with a user; providing the first physiological signal as an input to a first transformer model; providing the second physiological signal as an input to a second transformer model; providing an output of the first transformer model and an output of the second transformer model as inputs to a third transformer model; providing an output of the third transformer model to at least one BP estimation model; and generating an estimated BP value corresponding to the first physiological signal and the second physiological signal based on an output of the at least one BP estimation model.

The first physiological signal may include an electrocardiogram (ECG) signal associated with the user, and the second physiological signal may include a photoplethysmogram (PPG) signal associated with the user.

The first transformer model, the second transformer model, the third transformer model, and the at least one BP estimation model may be trained using a pre-training process and a user-specific training process, the pre-training process may be performed using a first training dataset corresponding to a plurality of users, and the user-specific training process may be performed based on a second training dataset corresponding to the user.

At least one of the pre-training process and the user-specific training process may be performed using a weighted contrastive loss function including a similarity metric.

The similarity metric may indicate a similarity between a first ground truth BP value corresponding to a first training sample and a second ground truth BP value corresponding to a second training sample.

The similarity metric may be used to cluster training samples included in at least one of the first training dataset and the second training dataset in an embedding space.

The at least one BP estimation model may include a systolic BP (SBP) estimation model and a diastolic BP (DBP) estimation model.

The method may further include providing the output of the third transformer model to the SBP estimation model; generating an estimated SBP value based on an output of the SBP estimation model; providing the output of the third transformer model to the DBP estimation model; and generating an estimated DBP value based on an output of the DBP estimation model, and the estimated BP value may include the estimated SBP value and the estimated DBP value.

In accordance with an aspect of the disclosure, an electronic device for performing cuffless blood pressure (BP) measurement, including: a first sensor configured to obtain a first physiological signal from a user; a second sensor configured to obtain a second physiological signal from the user; and at least one processor configured to: provide the first physiological signal as an input to a first transformer model; provide the second physiological signal as an input to a second transformer model; provide an output of the first transformer model and an output of the second transformer model as inputs to a third transformer model; provide an output of the third transformer model to at least one BP estimation model; and generate an estimated BP value corresponding to the first physiological signal and the second physiological signal based on an output of the at least one BP estimation model.

The first physiological signal may include an electrocardiogram (ECG) signal associated with the user, and the second physiological signal may include a photoplethysmogram (PPG) signal associated with the user.

The first transformer model, the second transformer model, the third transformer model, and the at least one BP estimation model may be trained using a pre-training process and a user-specific training process, the pre-training process may be performed using a first training dataset corresponding to a plurality of users, and the user-specific training process may be performed based on a second training dataset corresponding to the user.

At least one of the pre-training process and the user-specific training process may be performed using a weighted contrastive loss function including a similarity metric.

The similarity metric may indicate a similarity between a first ground truth BP value corresponding to a first training sample and a second ground truth BP value corresponding to a second training sample.

The similarity metric may be used to cluster training samples included in at least one of the first training dataset and the second training dataset in an embedding space.

The at least one BP estimation model may include a systolic BP (SBP) estimation model and a diastolic BP (DBP) estimation model.

The at least one processor may be further configured to: provide the output of the third transformer model to the SBP estimation model; generate an estimated SBP value based on an output of the SBP estimation model; provide the output of the third transformer model to the DBP estimation model; and generate an estimated DBP value based on an output of the DBP estimation model, and the estimated BP value may include the estimated SBP value and the estimated DBP value.

In accordance with an aspect of the disclosure, a non-transitory computer-readable medium stores instructions which, when executed by at least one processor of a device for performing cuffless blood pressure (BP) measurement, cause the device to: obtain a first physiological signal and a second physiological signal associated with a user; provide the first physiological signal as an input to a first transformer model; provide the second physiological signal as an input to a second transformer model; provide an output of the first transformer model and an output of the second transformer model as inputs to a third transformer model; provide an output of the third transformer model to at least one BP estimation model; and generate an estimated BP value corresponding to the first physiological signal and the second physiological signal based on an output of the at least one BP estimation model.

The first physiological signal may include an electrocardiogram (ECG) signal associated with the user, and the second physiological signal may include a photoplethysmogram (PPG) signal associated with the user.

The first transformer model, the second transformer model, the third transformer model, and the at least one BP estimation model may be trained using a pre-training process and a user-specific training process, the pre-training process may be performed using a first training dataset corresponding to a plurality of users, and the user-specific training process may be performed based on a second training dataset corresponding to the user.

At least one of the pre-training process and the user-specific training process may be performed using a weighted contrastive loss function including a similarity metric.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 illustrates example components of an electronic device in accordance with some embodiments of the disclosure;

FIG. 2A illustrates a first example (a pair of earbuds) of the electronic device in accordance with some embodiments of the disclosure;

FIG. 2B illustrates example components of the first example of the electronic device in accordance with some embodiments of the disclosure;

FIG. 3A illustrates a second example (a watch) of the electronic device in accordance with some embodiments of the disclosure;

FIG. 3B illustrates example components of the second example of the electronic device in accordance with some embodiments of the disclosure;

FIG. 4A illustrates a third example (a ring) of the electronic device in accordance with some embodiments of the disclosure;

FIG. 4B illustrates example components of the third example of the electronic device in accordance with some embodiments of the disclosure;

FIGS. 5A to 5E illustrate example structures of a sensor for detecting biometric information according to one or more example embodiments;

FIG. 6 illustrates an example system having a fourth example (a computer) of the electronic device with remotely located electronic devices and their sensors in accordance with some embodiments of the disclosure;

FIG. 7A illustrates an example structure of a model for performing cuffless BP monitoring in accordance with some embodiments of the disclosure;

FIG. 7B is a flowchart illustrating an example process for performing cuffless BP monitoring in accordance with some embodiments of the disclosure;

FIG. 8 illustrates an example overview of a training process for a model for performing cuffless BP monitoring in accordance with some embodiments of the disclosure;

FIG. 9A illustrates an example training process for training a model for performing cuffless BP monitoring in accordance with some embodiments of the disclosure;

FIG. 9B is a flowchart illustrating an example training process for training a model for performing cuffless BP monitoring in accordance with some embodiments of the disclosure; and

FIG. 10 is a flowchart illustrating an example process for performing cuffless BP monitoring in accordance with some embodiments of the disclosure.

### DETAILED DESCRIPTION

The terms as used in the disclosure are provided to merely describe specific embodiments, not intended to limit the scope of other embodiments. Singular forms include plural referents unless the context clearly dictates otherwise. The terms and words as used herein, including technical or scientific terms, may have the same meanings as generally understood by those skilled in the art. The terms as generally defined in dictionaries may be interpreted as having the same or similar meanings as or to contextual meanings of the relevant art. Unless otherwise defined, the terms should not be interpreted as ideally or excessively formal meanings. Even though a term is defined in the disclosure, the term should not be interpreted as excluding embodiments of the disclosure under circumstances.

The electronic device according to one or more embodiments may be one of various types of electronic devices. In some embodiments of the disclosure, the electronic devices may include a portable communication device (e.g., a smartphone), a computer device, a portable multimedia device, a portable medical device, a camera, or a wearable device. The electronic devices are not limited to those described above, in accordance with some other embodiments of the disclosure.

The disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B”, “at least one of A and B”, “at least one of A or B”, “A, B, or C”, “at least one of A, B, and C”, and “at least one of A, B, or C”, may include any one of, or all possible combinations of the items enumerated together in a corresponding one of the phrases. As used herein, such terms as “1st” and “2nd”, or “first” and “second” may be used to simply distinguish a corresponding component from another, and does not limit the components in other aspects (e.g., importance or order). It is to be understood that if an element (e.g., a first element) is referred to, with or without the term “operatively” or “communicatively”, as “coupled with”, “coupled to”, “connected with”, or “connected to” another element (e.g., a second element), it means that the element may be coupled with the other element directly (e.g., wiredly), wirelessly, or via a third element.

As discussed above, hypertension is a significant health concern and a major contributor to cardiovascular diseases. Accordingly, blood pressure (BP) readings, including systolic BP (SBP) values and diastolic BP (DBP) values, are important cardiovascular health indicators. Monitoring these can be vital in diagnosis and management of high blood pressure. However, many approaches to BP monitoring rely on cuff-based devices such as a sphygmomanometer, which works by restricting the blood flow on the arm and gradually releasing the pressure while monitoring the flow. These cuff-based devices may be used to accurately measure BP across a diverse population, but they may not be convenient for continuous monitoring, and therefore may not be widely adapted and used for continuous monitoring.

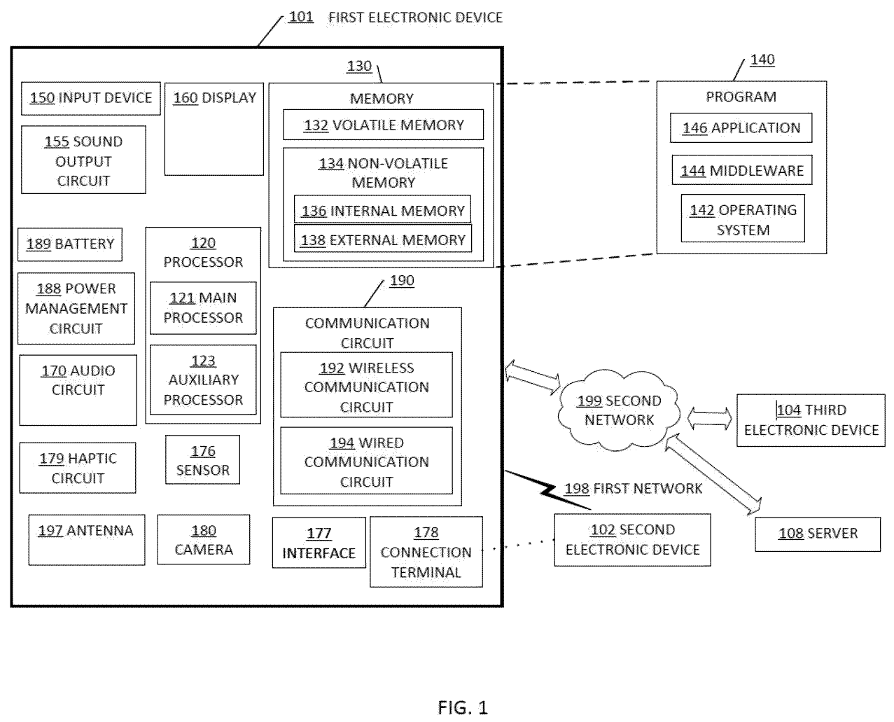

In order to perform cuffless BP monitoring, some approaches use statistical models based on other physiological signals such as electrocardiogram (ECG) and photoplethysmogram (PPG). These physiological signals may be easily measured by personal electronic devices (e.g., the one or more of the first electronic device 101, the earbuds 101A, the watch 101B, and the ring 101C discussed below with reference to FIGS. 1-6). However, these statistical model approaches are often inaccurate due to several simplifying assumptions made during feature extraction, for example the use of fixed feature windows (e.g., R-R interval), assuming uniform signal quality for ECG and PPG recording, and physiological assumptions such as assuming that PTT is directly related to BP while ignoring arterial stiffness or varying heart rates. In addition, many of these approaches rely on hand-crafted features (e.g., R-R interval, P-wave duration, pulse transit time (PTT), peak-to-peak interval, etc.) which must be manually selected and extracted.

Other approaches to cuffless BP monitoring use personalized deep leaning models, such as end-to-end deep neural network (DNN)-based BP prediction models. Unlike the statistical model approaches discussed above, it may not be necessary to choose what features to extract, because personalized DNN-based model may learn the correct features to extract in a data driven manner during training. However, unlike the statistical model approaches, this approach may use a large amount of training data. For example, some personalized models may require as much as twenty-seven hours of data from a subject in order to ensure accuracy with respect to that subject. One way to overcome this is to use a diverse training dataset to create a generalized DNN-based BP prediction model. This may reduce or eliminate the amount of subject-specific training data required. However, these generalized models often suffer from poor prediction accuracy because physiological measurements related to BP monitoring are very diverse, so collecting a training dataset that is diverse enough to ensure accuracy may be difficult.

Still further approaches to cuffless BP monitoring use transfer learning. An initial model may be pre-trained on a large and diverse dataset, and then may be fine-tuned using subject-specific data. This approach may greatly reduce the amount of subject-specific data needed to build highly accurate BP prediction models. Some approaches to transfer learning rely on prediction error (e.g., mean absolute error (MAE) and/or mean squared error (MSE)) as the metric for pre-training.

Accordingly, embodiments may relate to a novel end-to-end deep learning approach to cuffless blood pressure (BP) monitoring that employs a transformer architecture to analyze raw multimodal physiological signals, for example electrocardiogram (ECG) and photoplethysmogram (PPG) signals. The strong sequence modeling capabilities provided by transformers may make them useful for handling the intricacies of physiological time-series data. Embodiments may provide convenient and accurate BP monitoring without the need for manual feature engineering, thus enhancing model flexibility and adaptability.

Embodiments may relate to performing pre-training based on a relatively large and diverse dataset, followed by fine-tuning based on a smaller subject-specific dataset. For example, in some embodiments, the pre-training may be performed using a dataset corresponding to approximately one hundred subjects, and the fine-tuning may be performed using a dataset corresponding to up to ten subjects, but embodiments are not limited thereto. According to embodiments, the pre-training may be performed using one or more of a weighted contrastive learning (WCL) loss and a mean absolute error (MAE) loss. This may allow for fine-tuning using individual data to improve personalization while retaining high accuracy.

Accordingly, embodiments may apply end-to-end transformer architectures for personalized, cuffless BP monitoring using multimodal physiological signals. By leveraging end-to-end learning and transfer learning, embodiments may provide a scalable and personalized approach to cuffless BP monitoring with significant implications for personalized healthcare.

Embodiments of the disclosure may be implemented in or by the following example electronic devices shown in FIG. 1 to FIG. 6.

FIG. 1 illustrates example components of the electronic device in accordance with some embodiments of the disclosure.

In FIG. 1, a first electronic device 101 may communicate with a second electronic device 102 via a first network 198 (e.g., a short-range wireless communication network), or a third electronic device 104 or a server 108 via a second network 199 (e.g., a long-range wireless communication network). In one embodiment, the first electronic device 101 may communicate with the third electronic device 104 via the server 108. Throughout the disclosure, the first electronic device 101 is referred to as ‘the electronic device 101.’ Hereinafter, examples of components of the electronic device 101 are described. Those components of the electronic device 101 may be also included in at least one of the second electronic device 102 and the third electronic device 104.

In one embodiment, the electronic device 101 may include a processor 120, memory 130, an input device 150, a sound output circuit 155, a display 160, an audio circuit 170, a sensor 176, an interface 177, a haptic circuit 179, a camera 180, a power management circuit 188, a battery 189, a communication circuit 190, a subscriber identification module (SIM) 196, or an antenna 197.

In some embodiments, at least one (e.g., the display 160 or the camera 180) of the components may be omitted from the electronic device 101, or one or more other components may be added in the electronic device 101. In some embodiments, some of the components may be implemented as single integrated circuitry. For example, the sensor 176 (e.g., a fingerprint sensor, an iris sensor, or an illuminance sensor) may be implemented as embedded in the display 160 (e.g., a display).

The processor 120 may execute, for example, software (e.g., a program 140) to control at least one other component (e.g., a hardware or software component) of the electronic device 101 coupled with the processor 120, and may perform various data processing or computation. In one embodiment, as at least part of the data processing or computation, the processor 120 may load a command or data received from another component (e.g., the sensor 176 or the communication circuit 190) in volatile memory 132, process the command or the data stored in the volatile memory 132, and store resulting data in non-volatile memory 134. In one embodiment, the processor 120 may include a main processor 121 (e.g., a central processing unit (CPU) or an application processor (AP)), and an auxiliary processor 123 (e.g., a graphics processing unit (GPU), an image signal processor (ISP), a sensor hub processor, or a communication processor (CP)) that is operable independently from, or in conjunction with, the main processor 121. Additionally or alternatively, the auxiliary processor 123 may be adapted to consume less power than the main processor 121, or to be specific to a specified function. The processor 120 may refer to or correspond to one or more processors. For example, the electronic device 101 may include two or more processors like the processor 120.

The auxiliary processor 123 may be implemented as separate from, or as part of the main processor 121. The auxiliary processor 123 may control at least some of functions or states related to at least one component (e.g., the display 160, the sensor 176, or the communication circuit 190) among the components of the electronic device 101, instead of the main processor 121 while the main processor 121 is in an inactive (e.g., sleep) state, or together with the main processor 121 while the main processor 121 is in an active state (e.g., executing an application). In one embodiment, the auxiliary processor 123 (e.g., an image signal processor or a communication processor) may be implemented as part of another component (e.g., the camera 180 or the communication circuit 190) functionally related to the auxiliary processor 123.

The memory 130 may store various data used by at least one component (e.g., the processor 120 or the sensor 176) of the electronic device 101. The various data may include, for example, software (e.g., the program 140) and input data or output data for a command related thereto. The memory 130 may include the volatile memory 132 or the non-volatile memory 134. The program 140 may be stored in the memory 130 as software, and may include, for example, an operating system (OS) 142, middleware 144, or an application 146.

One or more embodiments of the disclosure may be implemented as software (e.g., the application 146, the middleware 144, the operating system) including one or more instructions that are stored in the memory 130 (a storage medium) that is readable by the electronic device 101. For example, the processor 120 of the electronic device 101 may invoke at least one of the one or more instructions stored in the memory 130, and execute the at least one of the one or more instructions, with or without using one or more other components under the control of the processor 120. This allows the electronic device 101 to be operated to perform at least one function according to the at least one instruction invoked. The one or more instructions may include a code generated by a compiler or a code executable by an interpreter. The memory 130, which may be a machine-readable storage medium, may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the memory 130 (the storage medium) and where the data is temporarily stored in the memory 130.

In some embodiments, functions related to artificial intelligence (AI) are operated by the processor 120 (or the main processor 121 or the auxiliary processor 123) and the memory 130. The processor 120 (or the main processor 121 or the auxiliary processor 123) may include or may correspond to a general-purpose processor, such as a CPU, an application processor, or a digital signal processor (DSP), a graphics-dedicated processor, such as a graphics processing unit (GPU) or a vision processing unit (VPU), or an artificial intelligence-dedicated processor, such as a neural processing unit (NPU). The processor 120 (or the main processor 121 or the auxiliary processor 123) may control input data to be processed according to predefined operation rules or artificial intelligence models, which are stored in the memory 130. Alternatively, the processor 120 (or the main processor 121 or the auxiliary processor 123) may be an artificial intelligence-dedicated processor including a hardware structure specialized for processing of a particular artificial intelligence model.

The predefined operation rules or the artificial intelligence models may be made through training. Here, the statement of being made through training means that a basic artificial intelligence model may be trained by a learning algorithm by using a large number of training data, thereby making a predefined operation rule or an artificial intelligence model, which is configured to perform a desired characteristic (or purpose). Such training may be performed in a device itself, in which artificial intelligence according to the disclosure is performed, or may be performed via a separate server or a separate system. Examples of the learning algorithm may include, but are not limited to, supervised learning, unsupervised learning, semi-supervised learning, and reinforcement learning.

The artificial intelligence model may include a plurality of neural network layers. Each of the plurality of neural network layers has a plurality of weight values and performs neural network calculations through calculations between a calculation result of a previous layer and the plurality of weight values. The plurality of weight values of the plurality of neural network layers may be optimized by a training result of the artificial intelligence model. For example, the plurality of weight values may be updated to minimize a loss value or a cost value, which is obtained from the artificial intelligence model during the process of training. An artificial neural network may include a DNN, and examples of the artificial neural network may include, but are not limited to, a random forest model, a convolutional neural network (CNN), a DNN, a recurrent neural network (RNN), a restricted Boltzmann machine (RBM), a deep belief network (DBN), a bidirectional recurrent deep neural network (BRDNN), and deep Q-Networks.

The input device 150 may receive a command or data to be used by other components (e.g., the processor 120) of the electronic device 101, from the outside (e.g., a user) of the electronic device 101. The input device 150 may include, for example, a microphone, a mouse, or a keyboard.

The sound output circuit 155 may output sound signals to the outside of the electronic device 101. The sound output circuit 155 may include, for example, a speaker or a receiver. The speaker may be used for general purposes, such as playing multimedia or playing record, and the receiver may be used for incoming calls. In one embodiment, the receiver may be implemented as separate from, or as part of the speaker.

The display 160 may visually provide information to the outside (e.g., a user) of the electronic device 101. The display 160 may include, for example, a display, a hologram device, or a projector and control circuitry to control a corresponding one of the display, hologram device, and projector. In one embodiment, the display 160 may include touch circuitry adapted to detect a touch, or sensor circuitry (e.g., a pressure sensor) adapted to measure the intensity of force incurred by the touch.

The audio circuit 170 may convert a sound into an electrical signal and vice versa. In one embodiment, the audio circuit 170 may obtain the sound via the input device 150, or output the sound via the sound output circuit 155 or a headphone of an external electronic device (e.g., an electronic device 102) directly (e.g., wiredly) or wirelessly coupled with the electronic device 101.

The sensor 176 may detect an operational state (e.g., power or temperature) of the electronic device 101 or an environmental state (e.g., a state of a user) external to the electronic device 101, and then generate an electrical signal or data value corresponding to the detected state. In one embodiment, the sensor 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. In one embodiment, the interface 177 may include, for example, a high definition multimedia interface (HDMI), a universal serial bus (USB) interface, a secure digital (SD) card interface, or an audio interface.

A connection terminal 178 may include a connector via which the electronic device 101 may be physically connected with the external electronic device (e.g., the electronic device 102). In one embodiment, the connection terminal 178 may include, for example, a HDMI connector, a USB connector, a SD card connector, or an audio connector (e.g., a headphone connector),

The haptic circuit 179 may convert an electrical signal into a mechanical stimulus (e.g., a vibration or a movement) or electrical stimulus which may be recognized by a user via his tactile sensation or kinesthetic sensation. In one embodiment, the haptic circuit 179 may include, for example, a motor, a piezoelectric element, or an electric stimulator.

The camera 180 may capture a still image or moving images. In one embodiment, the camera 180 may include one or more lenses, image sensors, image signal processors, or flashes.

The power management circuit 188 may manage power supplied to the electronic device 101. In one embodiment, the power management circuit 188 may be implemented as at least part of, for example, a power management integrated circuit (PMIC).

The battery 189 may supply power to at least one component of the electronic device 101. In one embodiment, the battery 189 may include, for example, a primary cell which is not rechargeable, a secondary cell which is rechargeable, or a fuel cell.

The communication circuit 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication circuit 190 may include one or more communication processors that are operable independently from the processor 120 (e.g., the application processor (AP)) and supports a direct (e.g., wired) communication or a wireless communication. In one embodiment, the communication circuit 190 may include a wireless communication circuit 192 (e.g., a cellular communication circuit, a short-range wireless communication circuit, or a global navigation satellite system (GNSS) communication circuit) or a wired communication circuit 194 (e.g., a local area network (LAN) communication circuit or a power line communication (PLC) module). A corresponding one of these communication circuits may communicate with the external electronic device via the first network 198 (e.g., a short-range communication network, such as Bluetooth™, wireless-fidelity (Wi-Fi) direct, or infrared data association (IrDA)) or the second network 199 (e.g., a long-range communication network, such as a cellular network, the Internet, or a computer network (e.g., LAN or wide area network (WAN)). These various types of communication circuits may be implemented as a single component (e.g., a single chip), or may be implemented as multi components (e.g., multi chips) separate from each other. The wireless communication circuit 192 may identify and authenticate the electronic device 101 in a communication network, such as the first network 198 or the second network 199, using subscriber information (e.g., international mobile subscriber identity (IMSI)) stored in the subscriber identification module 196.

The antenna 197 may transmit or receive a signal or power to or from the outside (e.g., the external electronic device) of the electronic device 101. In one embodiment, the antenna 197 may include one or more antennas, and, therefrom, at least one antenna appropriate for a communication scheme used in the communication network, such as the first network 198 or the second network 199, may be selected, for example, by the communication circuit 190 (e.g., the wireless communication circuit 192). The signal or the power may then be transmitted or received between the communication circuit 190 and the external electronic device via the selected at least one antenna.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

In one embodiment, commands or data may be transmitted or received between the electronic device 101 and the external electronic device 104 via the server 108 coupled with the second network 199. Each of the electronic devices 102 and 104 may be a device of a same type as, or a different type, from the electronic device 101. In one embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, or client-server computing technology may be used, for example.

The electronic device according to various embodiments may be one of various types of electronic devices. The electronic devices may include, for example, a portable communication device (e.g., a smart phone), a computer device, a portable multimedia device, a portable medical device, a camera, a wearable device, or a home appliance. In one embodiment of the disclosure, the electronic devices are not limited to those described above.

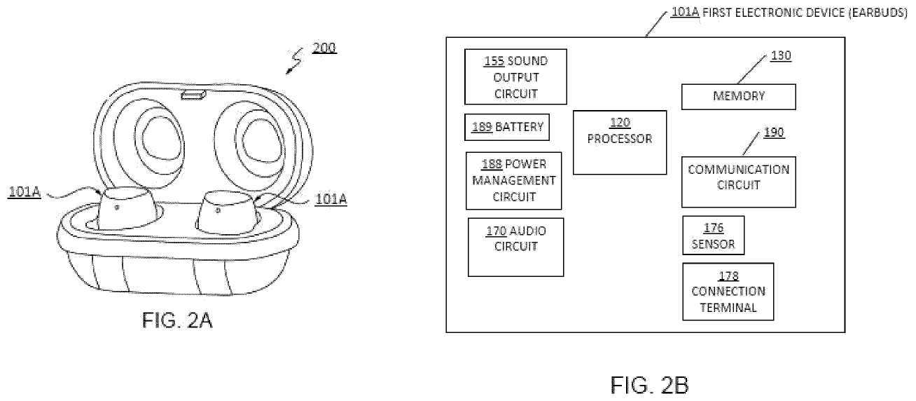

FIG. 2A illustrates an example of the electronic device 101, which is a pair of earbuds 101A with a power supply device 200. FIG. 2A also illustrates an example of the power supply device 200. In some embodiments, the power supply device 200 can have different forms, such as a wall charger. FIG. 2B illustrates that the earbuds 101A may include the processor 120, the memory 130, the sound output circuit 155, the audio circuit 170, the sensor 176, the connection terminal 178, the power management circuit 188, the battery 189, and the communication circuit 190. Those components shown in FIG. 2B are examples, that is, some of those components may be omitted or other components (for example, shown in FIG. 1) may be added to the earbuds 101A.

In some embodiments, the earbuds 101A may be paired with a computer, an electronic device, or a mobile device that corresponds to the first electronic device 101 shown in FIG. 1. That is, any data processed by the earbuds 101A can be shown on the display 160 of the first electronic device 101. In some embodiments, some operations can be shared between the earbuds 101A and the first electronic device 101.

In FIG. 2A, when one or more of the earbuds 101A are not in use, the earbuds 101A may be accommodated and stored in the power supply device 200, and when the earbuds 101A are in use, they may be worn and brought into close proximity to a part of the user's body (e.g., ears). In one embodiment, the earbuds 101A may be configured as a pair to be worn on both ears of the user and brought into close proximity.

In one embodiment, the earbuds 101A may output an audio signal (e.g., via the sound output circuit 155) when worn and in close proximity to a part of the user's body. In one embodiment, at least one of a right earbud and a left earbud may output an audio signal using wireless data transmission and reception with an electronic device (such as a mobile phone), for example, configured to play a song to be transmitted to the earbuds 101A.

For example, the wireless data transmission and reception path may be based on or may correspond to a Bluetooth communication scheme, a route for a Bluetooth low energy (BLE) communication scheme, and a route for a ultra-wide band (UWA) communication scheme. The wireless data transmission and reception path may include at least one of a path for a wireless fidelity (Wi-Fi) direct communication technique and a path for a mobile communication technique (e.g., long term evolution (LTE) sidelink, etc.).

In one embodiment, only one of the earbuds 101A may create the communication path with the electronic device. For example, a first earbud may output an audio signal based on the audio data of the electronic device received through the communication path (the wireless data transmission and reception path). When the electronic device is connected to the right earbud, the electronic device or the right earbud provides information about the communication path to the left earbud so that the left earbud can output the audio signal. The left earbud can receive data transmitted to the right earbud based on the information about the communication path and output the audio signal.

For example, the left earbud can receive data transmitted to the right earbud by monitoring information about the communication path. In one embodiment, the right earbud connected to the electronic device may be referred to as a master device, and the left earbud not connected to the electronic device may be referred to as a slave device. In one embodiment, the master device and the slave device among the pair of the earbuds 101A may be changed. In one embodiment, at least one of the earbuds 101A may transmit data to the electronic device. For example, the data may include information for controlling the audio signal output through the earbuds 101A (e.g., information for playing a sound source, information for pausing the sound source, information for stopping the sound source, information for controlling the volume of the sound source, and information for selecting the sound source.

In FIG. 2A, the power supply device 200 may have a structure that can be opened and closed. In one embodiment, in response to the operation of opening the power supply device 200, the power supply device 200 may perform a triggering operation for Bluetooth pairing with the earbuds 101A. In one embodiment, the earbuds 101A is generally small in size and may include a rechargeable battery (e.g., the battery 189) to provide mobility. Accordingly, the earbuds 101A may be stored connected to a separate power supply device 200 to prevent loss while the earbuds 101A is not in use. In one embodiment, the earbuds 101A may be connected to the power supply device 200 and be able to charge the battery while being stored. In one embodiment, the earbuds 101A may include a sensor (e.g., the sensor 176) corresponding to a sensor of the power supply device 200. For example, the power supply device 200 may include a Hall sensor (or hall IC) and the earbuds 101A may include a magnet (for example, included in the sensor 176). In one embodiment, when the earbuds 101A is accommodated in the power supply device 200, the Hall sensor of the power supply device 200 can recognize a magnet in the earbuds 101A.

In one embodiment, the earbuds 101A may include at least one conductive pin pad (e.g., the connection terminal 178) on the outside. In one embodiment, the power supply device 200 may include at least one externally conductive pin (e.g., a conductive terminal). The conductive pin pad of the earbuds 101A and the conductive pin of the power supply 200 may be arranged to physically contact each other while the earbuds 101A is connected to the power supply 200. In one embodiment, when the earbuds 101A is connected to the power supply device 200, the conductive pin of the earbuds 101A and the conductive pin of the power supply device 200 may be in contact and electrically connected. In one embodiment, by identifying a conductive pin contact, the earbuds 101A or the power supply 200 can determine whether the earbuds 101A is connected to the power supply 200. In one embodiment, the earbuds 101A may detect the amount of light reflected from the power supply device 200 through a proximity sensor (e.g., included in the sensor 176).

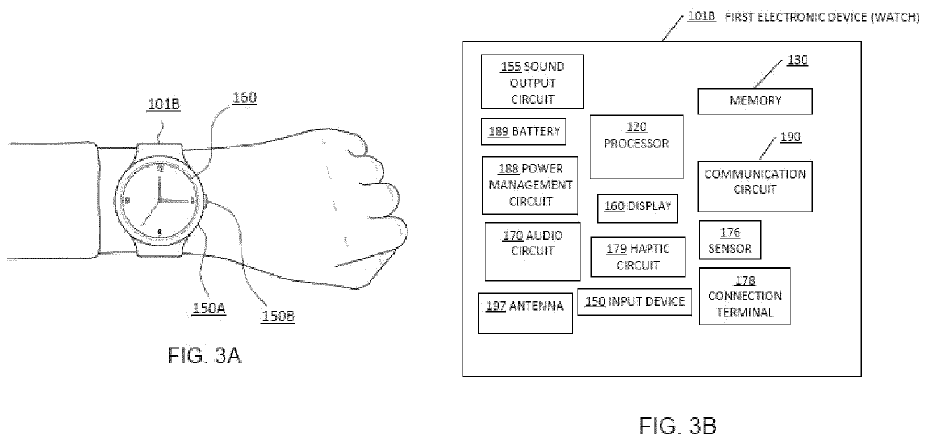

FIG. 3A illustrates a second example of the electronic device 101, which is a watch 101B. FIG. 3B illustrates that the watch 101B may include multiple components, such as the processor 120, the memory 130, the input device 150 (e.g., the input devices 150A and 150B), the sound output circuit 155, the display 160, the audio circuit 170, the sensor 176, the connection terminal 178, the haptic circuit 179, the power management circuit 188, the battery 189, and the communication circuit 190. Those components shown in FIG. 3B are examples, that is, some of those components may be omitted or other components (for example, shown in FIG. 1) may be added to the watch 101B.

In some embodiments, the watch 101B may be paired with a computer, an electronic device, or a mobile device that corresponds to the first electronic device 101 shown in FIG. 1. That is, any data processed by the watch 101B can be shown on the display 160 of the first electronic device 101. In some embodiments, some operations can be shared between the watch 101B and the first electronic device 101.

In FIG. 3A, the watch 101B may include a first input device 150A, a second input device 150B, and the display (touch screen) 160. The watch 101B may be implemented as a smart watch. The processor 120 of the watch 101B may control the overall operation of the watch 101B.

The watch 101B may display data or information through the display 160. The watch 101B may display data stored in the memory 130 through the display 160. For example, the watch 101B may display data or information provided by at least one application stored in the memory 130 through the display 160. Further, the watch 101B may display an execution screen or an execution state of at least one application through the display 160.

In one embodiment, the display 160 may display a user interface for performing a function of the watch 101B. For example, the display 160 may display an execution screen of at least one application stored in the watch 101B. Further, the display 160 may simultaneously display execution screens of a plurality of applications stored in the watch 101B.

In one embodiment, through the display 160, the watch 101B may identify at least one of a touch input, drag input (or touch-drag input), swipe input, and pinch input on the display 160.

The first input device 150A may generate an input signal in response to an input to the watch 101B. For example, the first input device 150A may be implemented in the form of a bezel of a watch. The user may rotate the first input device 150A in a first direction (for example, a clockwise direction) or a second direction (for example, a counterclockwise direction), and the watch 101B may identify the input corresponding to the rotation.

The watch 101B may further include the second input device 150B. For example, the second input device 150B may be implemented in the form of a watch stem. The user may rotate the second input device 150B and press the second input device 150B, and the watch 101B may identify the input corresponding to the rotation or pressing.

The watch 101B may further include the sound output circuit 155 (e.g., a speaker). For example, the watch 101B may provide information in the form of a voice or a sound through the sound output circuit 155.

In some embodiments, the execution screen may be a screen on which a corresponding application is executed and that indicates an execution state. That is, the corresponding application has already been executed or is being executed.

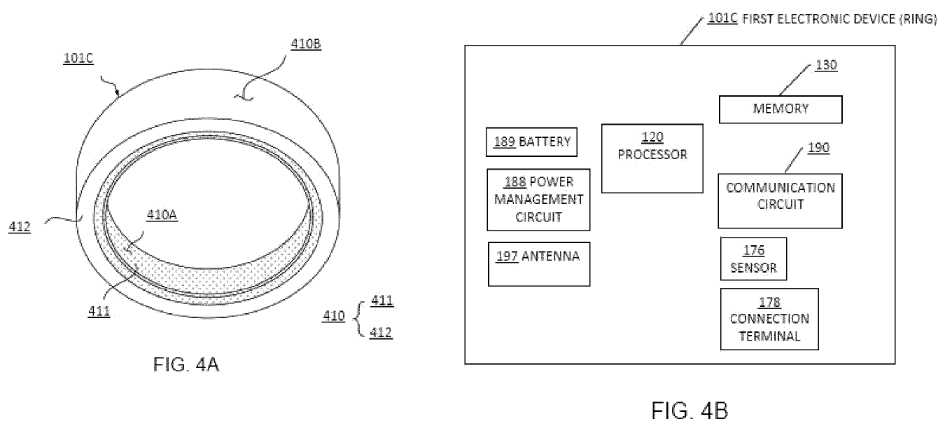

FIG. 4A illustrates a third example of the electronic device 101, which is a ring 101C. FIG. 4B illustrates that the ring 101C may include multiple components.

In FIG. 4A, the ring 101C may include a frame 410. According to an embodiment, the ring 101C may be worn by a user. For example, the ring 101C may be configured to obtain information related to the user through the body part. For example, the ring 101C may provide the user with information indicating a state of the user, based on obtaining the information related to the user. For example, the ring 101C may be configured to display information indicating the state of the user through a display of the ring 101C and/or an external electronic device (e.g., the second electronic device 102 or the third electronic device 104 of FIG. 1) connected to the ring 101C, thereby providing the user with the information indicative of the state of the user. The ring 101C may provide the user with information related to the user wearing the ring 101C via the external electronic device connected to the ring 101C.

In some embodiments, the ring 101C may be paired with a computer, an electronic device, or a mobile device that corresponds to the first electronic device 101 shown in FIG. 1. That is, any data processed by the ring 101C can be shown on the display 160 of the first electronic device 101. In some embodiments, some operations can be shared between the ring 101C and the first electronic device 101.

For example, the body part of the user (on which the ring 101C is worn) may be the user's finger. For example, the frame 410 of the ring 101C may have a ring shape, such that the ring 101C may be worn on the user's finger. However, the disclosure is not limited thereto. The ring 101C, which may be referred to as a wearable device, may have a shape corresponding to the body part of the user.

According to an embodiment, the frame 410 may include a first surface 410A (e.g., an inner surface) facing the body part of the user while the ring 101C is worn on the body part of the user, and a second surface 410B (e.g., an outer surface) opposite to the first surface 410A. The body part may be one of the user's fingers. For example, when the ring 101C is worn by the user, at least a portion of the first surface 410A may contact the body part of the user. For example, the first surface 410A may surround the body part of the user wearing the ring 101C. For example, the first surface 410A may cover the body part of the user wearing the ring 101C. For example, the first surface 410A may be configured such that the ring 101C is fastened to the body part by pressurizing the body part of the user when the ring 101C is worn by the user.

For example, the second surface 410B may be an external appearance of ring 101C together with the first surface 410A. For example, the second surface 410B may be a ring-shaped frame 410 together with the first surface 410A. The second surface 410B (opposite to the first surface 410A) may be farthest from the body part. For example, the first surface 410A may be an inner circumferential surface of the frame 410. The second surface 410B (opposite to the first surface 410A) may be an outer circumference surface of the frame 410.

Here, the ring 101C has been described as being worn on a body part of the user, but the disclosure is not limited thereto. The term “body part” is used to describe the body part of the user on which the ring 101C is worn and is not intended to limit the body part of the user on which the ring 101C is worn or a positional relationship between the body part and the ring 101C thereto. For example, the body part may be one of the user's fingers, but is not limited thereto.

According to an embodiment, the frame 410 may include a first frame 411 defining the first surface 410A, and a second frame 412 defining the second surface 410B and coupled to the first frame 411.

For example, the first frame 411 may be a portion of the frame 410 including the first surface 410A. For example, when the ring 101C is worn by the user, the first frame 411 may be in contact with the body part of the user. The first frame 411 may include at least one of silicon, epoxy, and acryl, but is not limited thereto.

For example, the second frame 412 may surround the first frame 411. For example, the second frame 412 may support the first frame 411. For example, the second frame 412 may form the outer appearance of the frame 410 together with the first frame 411. For example, the second frame 412 may be a portion of the frame 410 including a second surface 410B opposite to the first surface 410A. The second frame 412 may include at least one of metal and titanium, but is not limited thereto. The frame 410 of the ring 101C may include the first frame 411 and the second frame 412 including different materials, thereby providing a user with various user experiences.

In FIG. 4B, the ring 101C may include multiple components in the frame 410 to perform functions of the ring 101C. For example, the ring 101C may include the processor 120, the memory 130, the sensor 176, the battery 189, the communication circuit 190, the power management circuit 188, and the antenna 197. For example, those components of the ring 101C may be included in the first frame 411. The components included in the ring 101C are not limited to the above-described components.

In some embodiments, the sensor 176 may include at least one of a temperature sensor, a proximity sensor, a motion sensor, or a pressure sensor. Further, the sensor 176 may include a light emitter facing the first surface 410A of the frame 410 and a light receiver spaced apart from the light emitter. In some embodiments, the sensor 176 is configured to detect biometric information about the user.

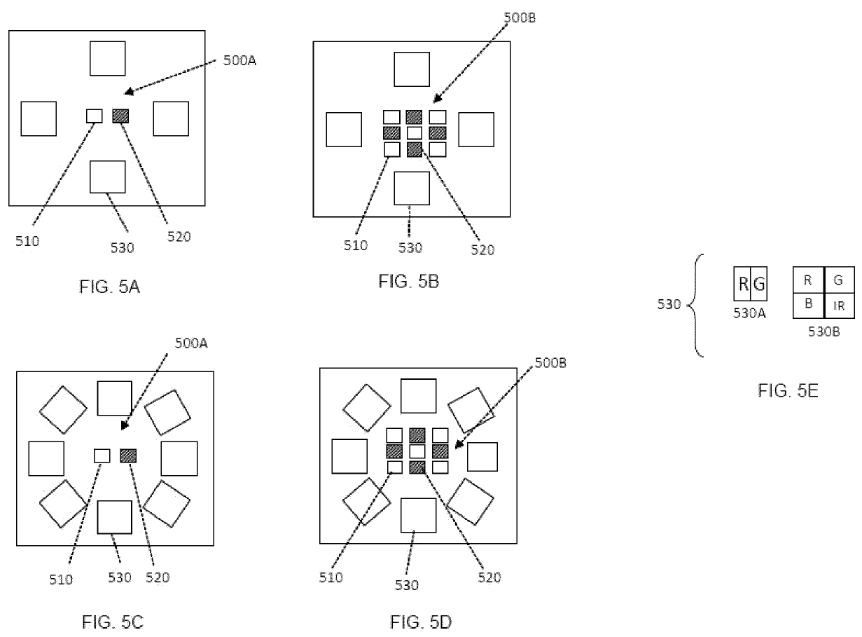

FIGS. 5A to 5E illustrate example structures of a sensor for detecting biometric or physiological information according to one or more example embodiments. The sensor of FIGS. 5A to 5E may correspond to the sensor 176 of FIGS. 1, 2B, 3B, and 4B.

In FIG. 5A, a light emission portion 500A may include a first light emission element 510 and a second light emission element 520. A plurality of light-receiving devices 530 may be provided around or near the light emission portion 500A. For example, four light-receiving devices 530 may be provided, one on each of the upper, lower, left, and right sides of the light emission portion 500A. In other words, the light-receiving devices 530 may be arranged in at least four positions around the light emission portion 500A. However, embodiments of the present disclosure are not limited thereto; for example, two light-receiving devices 530 may be provided, one on each of the opposite sides of the light emission portion 500A. Photodiodes, phototransistors, or charge-coupled devices (CCDs) may be used as the light-receiving devices 530. The light emission portion 500A and the light-receiving devices 530 may be provided on or in a substrate (e.g., a PCB).

In FIG. 5A, the light emission portion 500A may include a plurality of first light emission elements 510 and a plurality of second light emission elements 520, and FIG. 5Billustrates an example thereof.

In FIG. 5B, a light emission portion 500B may include a two-dimensional (2D) array in which a plurality of first light emission elements 510 and a plurality of second light emission elements 520 are mixed.

Also, according to another exemplary embodiment, the examples shown in FIGS. 5A-5B may be modified to arrange a plurality of light-receiving devices 530 in an annular array around the light emission portions 500A and 500B, and FIGS. 5C-5D illustrate examples thereof.

Each of the light-receiving devices 530 shown in FIGS. 5A-5D may be implemented as a single photodiode, or as two or more photodiodes as shown in FIG. 5E. Referring to FIG. 5E, a light-receiving device 530A may include two light-receiving devices configured to receive optical signals having different wavelength ranges, such as an optical signal in a red R region, and an optical signal in a green G region. In this case, one of the first emission element 510 and the second light emission element 520 may be a red light source, and the other one may be a green light source. However, the light emission wavelengths of the first emission element 510 and the second light emission element 520 and the light-receiving device 530A are not limited to these specific wavelengths and may be varied in various ways.

The structure of the light-receiving device 530 may be modified to have the structure of a light-receiving device 530B as illustrated in FIG. 5E. The light-receiving device 530B may include four light-receiving devices configured to receive optical signals of different wavelength ranges. For example, the four light-receiving devices may be configured to receive optical signals in the wavelength ranges of red R, green G, blue B, and infrared IR light, respectively. The light emission wavelengths of the light emission portions 500A and 500B may be configured to correspond with the wavelength ranges of the received optical signals.

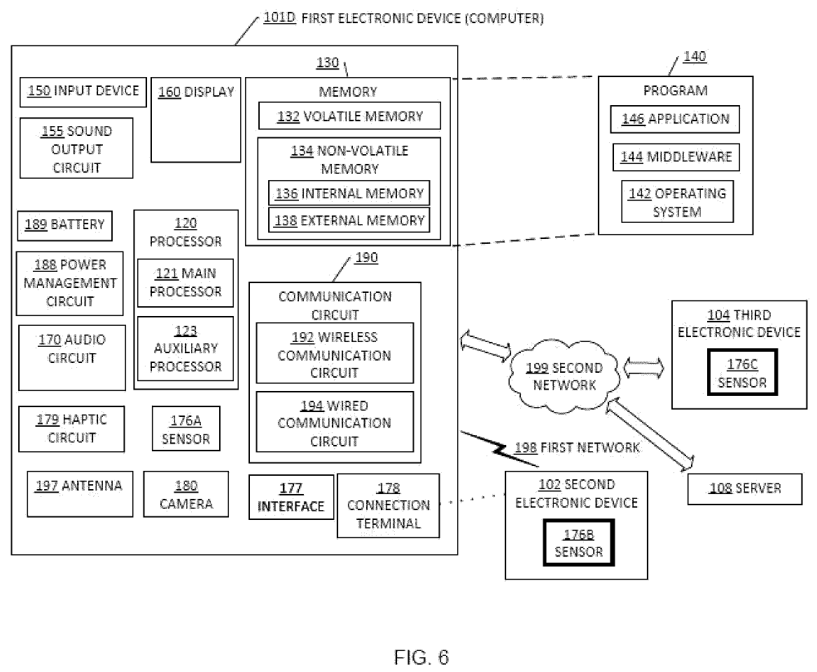

FIG. 6 illustrates an example system having a fourth example (e.g., a computer) of the electronic device with remotely located electronic devices and their sensors in accordance with some embodiments of the disclosure. The electronic device (computer) 101D may include multiple components such as the processor 120 and the memory 130, which are described in the above paragraphs. Other components are described above with respect to FIG. 2.

In FIG. 6, the remotely located electronic devices, namely, the second electronic device 102 and the third electronic device 103 may include their respective sensors, the sensor 176B and the sensor 176C, which may respectively correspond to the sensor illustrated in FIGS. 5A-5E.

In one case where a user is located at or near the second electronic device 102, the sensor 176B of the second electronic device 102 may be used to detect signals (e.g., biometric signals or physiological signals) from the user. Then, the second electronic device 102 may transmit the detected signals to the electronic device (computer) 101D over the first network 198. Next, the transmitted signals may be (temporarily or permanently) stored in the memory 130 and the processor 120 may perform various functions regarding the signals originally generated from the sensor 176B of the second electronic device 102.

Similarly, in another case where a user is located at or near the third electronic device 104, the sensor 176C of the third electronic device 104 may be used to detect signals (e.g., biometric signals) from the user. Then, the third electronic device 104 may transmit the detected signals to the electronic device (computer) 101D over the second network 199. Next, the transmitted signals may be (temporarily or permanently) stored in the memory 130 and the processor 120 may perform various functions regarding the signals originally generated from the sensor 176C of the third electronic device 104.

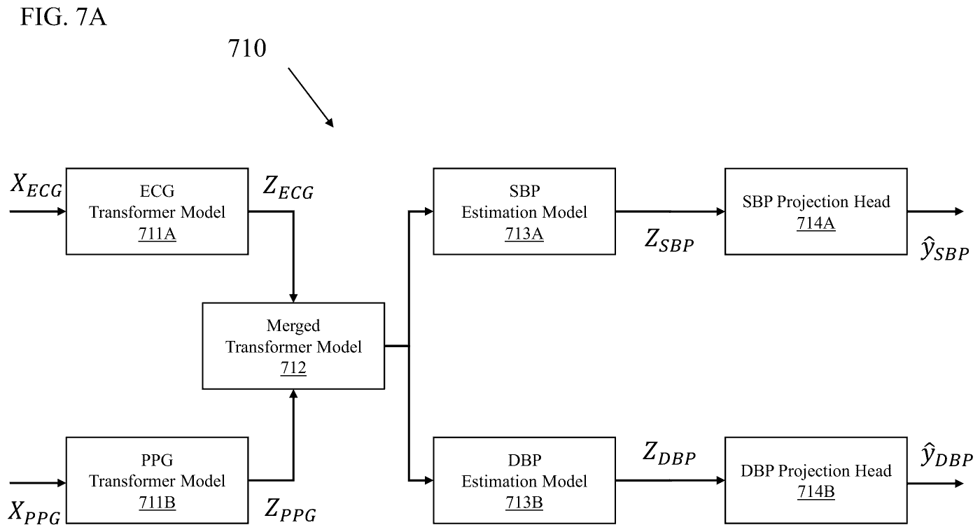

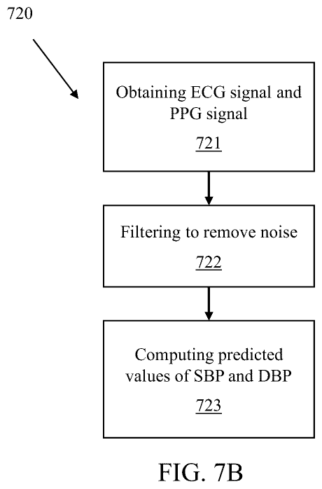

FIG. 7A is a block diagram illustrating an example of a model for performing cuffless BP monitoring, according to embodiments. According to embodiments, a model 710 may have a transformer-based architecture, and may generate predicted BP values based on multimodal inputs. For example, as shown in FIG. 7A, the model 710 may include an ECG transformer model 711A, a PPG transformer model 711B, a merged transformer model 712, an SBP estimation model 713A, a DBP estimation model 713B, an SBP projection head 714A, and a DBP projection head 714B.

According to embodiments, the ECG transformer model 711A, the PPG transformer model 711B, and the merged transformer model 712 may be time-series transformer (TST) models, which may be a type of network used to process sequential data and assist in capturing temporal dependencies and patterns. In some embodiments, the ECG transformer model 711A, the PPG transformer model 711B, and the merged transformer model 712 may be PatchTST models, but embodiments are not limited thereto, and any transformer architecture may be used.

According to embodiments, the SBP estimation model 713A and the DBP estimation model 713B may be separate multi-layer perceptrons (MLP), but embodiments are not limited thereto. For example, in some embodiments the SBP estimation model 713A and the DBP estimation model 713B may be any other type of network, for example CNNs or RNNs.

According to embodiments, the SBP projection head 714A and the DBP projection head 714B may be projection networks or projection layers, for example fully-connected (FC) layers, but embodiments are not limited thereto.

FIG. 7B is a flowchart illustrating an example process for performing cuffless BP monitoring, according to embodiments. According to embodiments, process 720 shown in FIG. 7B may be performed by any of the elements discussed above with reference to FIGS. 1-6 and 7A. In some embodiments, the process 720 shown in FIG. 7B may be referred to as an inference process, an inference time, an inference phase, a deployment time, or a deployment phase.

As shown in FIG. 7B, at operation 721, the process 720 may include obtaining multimodal physiological data from a subject. For example, in some embodiments operation 721 may include using one or more of the one or more of the first electronic device 101, the earbuds 101A, the watch 101B, and the ring 101C discussed above with reference to FIGS. 1-6 to obtain ECG signals x_{ECG} and PPG signals x_{PPG} using the sensor 176. In embodiments, the ECG signals x_{ECG} and PPG signals x_{PPG} may be obtained for the same predetermined frame length from a sensory device.

As further shown in FIG. 7B, at operation 722, the ECG signals x_{ECG} and PPG signals x_{PPG} may be aligned and filtered to remove noise. However, in some embodiments the process 720 may not include operation 722.

As further shown in FIG. 7B, at operation 723 the ECG signals x_{ECG} and PPG signals x_{PPG} may be provided to the model 710 to obtain predicted BP values such as a predicted SBP value \hat{γ}_{SBP} and a predicted DBP value \hat{γ}_{DBP}.

For example, at operation 723, the ECG transformer model 711A and the PPG transformer model 711B may be used to independently process the ECG signals x_{ECG} and the PPG signals X_{PPG} to extract features into a similar embedding space so that they can be combined. In embodiments, the ECG transformer model 711A may generate ECG embeddings z_{ECG} based on the ECG signals x_{ECG}, and the PPG transformer model 711B may generate PPG embeddings z_{PPG} based on the PPG signals x_{PPG}. The ECG embeddings z_{ECG} and the PPG embeddings z_{PPG} may be combined, for example using concatenation or summing, and the combined embeddings may be provided as input to the merged transformer model 712, which may extract synergistic features that can only be extracted leveraging the information of both of the input modalities. The output of the merged transformer model 712 may be provided to separate branches for systolic blood pressure (SBP) and diastolic blood pressure (DBP). For example, the output of the merged transformer model 712 may be provided to the SBP estimation model 713A to generate SBP embeddings z_{SBP}, and may be provided to the DBP estimation model 713B to generate DBP embeddings z_{DBP}. Then, the projection heads may be used to project the embeddings to the predicted BP values. For example, in embodiments the SBP projection head 714A may generate a predicted SBP value \hat{γ}_{SBP} based on the SBP embeddings z_{SBP}, and the DBP projection head 714B may generate a predicted DBP value \hat{γ}_{DBP} based on the DBP embeddings z_{DBP}. In embodiments, the predicted BP values may then be passed along to any downstream applications that might be depending on them

Although examples are provided herein in which the multimodal physiological signals include the ECG signals x_{ECG} and PPG signals x_{PPG}, embodiments are not limited thereto. For example, according to embodiments the model 710 may be signal-agnostic, and may be applied to any other signals or modalities. For example, in some embodiments the model 710 may receive as inputs one or more of physiological signals such as heart rate variability (HRV), respiration rate, electroencephalogram (EEG), skin conductance, etc., behavioral measurements such as movement, gait, voice, stress, cognition, etc., and chemical measurements such as glucose level, sweat composition, etc.

As discussed above, a large subject-specific dataset may be used to train a model for performing cuffless BP monitoring, for example a training dataset including triplets of ECG readings, PPG readings and ground-truth BP readings of a particular subject, taken at multiple time points. Some approaches that use such datasets use a straightforward learning mechanism that attempts to directly predict the BP values, given the ECG and PPG signals as input. For example, these approaches may train the model using only an MAE loss function, as shown for example in Equation 1 below:

$${ℒ}_{MAE}^{( \cdot )} = \frac{1}{N} {\sum }_{i = 1}^{N} \vert {\hat{y}}_{i} - {y}_{i} \vert \qquad Equation 1$$

In Equation 1 above, ( ) may denote one of SBP and DBP for which the loss is being computed, N may denote a number of samples in the training dataset, γᵢ may denote a ground-truth SBP value or DBP value for a given sample i, and \hat{γ}ᵢ may denote a corresponding predicted SBP value or DBP value for the sample i. While MAE loss may be effective, it does not offer any regularization.

According to embodiments, in order to provide improved SBP and DBP prediction, a different type of loss, which may be referred to as weighted contrastive learning (WCL) loss, may be used during training in order to bring together representations of ECG and PPG signals that have similar SBP values and DBP values. According to embodiments, such representations then lead to a better SBP and DBP regression. An example of a WCL loss function is shown below in Equation 2.

$${ℒ}_{WCL}^{( \cdot )} = {\sum }_{i}^{N} \frac{- 1}{{\sum }_{j = 1}^{N} {S}_{ij}} {\sum }_{j = 1}^{N} {S}_{ij} \cdot log \frac{exp ( {z}_{i}^{T} \cdot {z}_{j} / {τ}_{w} )}{{\sum }_{k = 1}^{N} exp ( {z}_{i}^{T} \cdot {z}_{k} / {τ}_{w} )} \qquad Equation 2$$

In Equation 2, Sᵢⱼ may denote a similarity metric between two samples indexed by i and j, zᵢ may denote the SBP embedding or DBP embedding of the sample i, τ_{w} may govern how similarity decays with distance in the embedding space, and T may denote transposition. The WCL loss may be general in that it may be applied to the SBP embeddings and DBP embeddings separately.

In some embodiments, the similarity metric Sᵢⱼ may be computed according to Equation 3 below.

$${S}_{ij} = \{ \begin{matrix}exp ( - {( \frac{\vert {y}_{i} - {y}_{j} \vert}{{τ}_{s}} )}^{p} ) = {S}_{ij}^{*} & if {S}_{ij}^{*} > {T}_{s} \\ 0 & otherwise\end{matrix} . \qquad Equation 3$$

In Equation 3 above, zⱼ may denote the SBP embedding or DBP embedding of the sample j, τₛ may denote a temperature term that governs how similarity decays with distance in the label space, Tₛ may denote a threshold below which two samples are deemed dis-similar, and ρ may denote a power term that governs a tail decay of similarity in the label space. In embodiments, at least one of τₛ, Tₛ, ρ, and τ_{w} may be hyperparameters.

According to embodiments, WCL loss may differ from other loss functions based on how it handles the relationship between samples during training. In approaches based solely on loss functions such as MAE loss and MSE loss, the difference between a predicted result and a corresponding ground truth result are minimized, without considering the relationship between different samples in the dataset. In contrast, using WCL loss may allow samples which are similar to each other to be clustered together in the embedding space. For example, training based on WCL may force the model to group similar data points (e.g., ECG signals and PPG signals with similar ground-truth SBP values and DBP values), in a high-dimensional space (e.g., the latent space or embedding space). This clustering may indicate that the model recognizes these data points as being related, which may assist in providing more accurate predictions. For example, this may allow the model to shape the embedding space in a way that resembles the ground-truth space (e.g., the space including the ground-truth SBP values and DBP values).

As an example in which the sample i is not equal to the sample j, the SBP embedding z_{SBP}ⁱ of the sample i and the SBP embedding z_{SBP}^{j} of the sample j which have similar ground truth SBP values γ_{SBP} may be drawn closer to each other, and the higher the similarity S_{SBP}^{ij} is, the higher the attraction may be. However, if S_{SBP}^{ij}=0, then the SBP embeddings z_{SBP}ⁱ and z_{SBP}^{j} may be considered dissimilar, and therefore may be repelled from each other.

Although examples are discussed above in which the similarity metric Sᵢⱼ is computed using Equation 3, embodiments are not limited thereto. For example, according to embodiments the similarity metric Sᵢⱼ may be computed using any other type of similarity, for example cosine similarity, L1 norm similarity, L2 norm similarity, dynamic time warping (DTW), or Jaccard similarity.

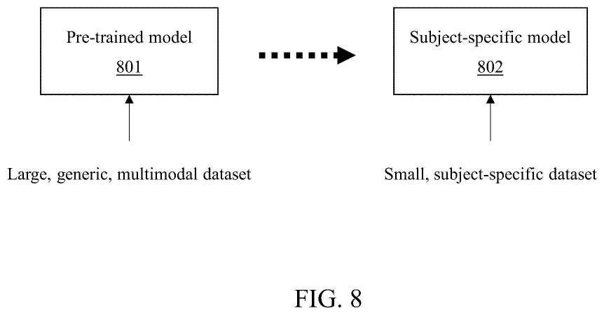

FIG. 8 is a block diagram illustrating an overview of a training process for training a model for performing cuffless BP monitoring, according to embodiments. As discussed above, some approaches to training a model for performing cuffless BP monitoring may require a large, subject-specific dataset to achieve accurate results. However, this involves collecting and annotating a large amount of physiological data such as ECG, PPG and BP readings from a particular subject. Therefore, according to embodiments, a pre-training process may be performed using a large and diverse multimodal dataset to obtain a pre-trained model 801. For example, the pre-training dataset may include PPG and ECG samples from many patients. Then, a fine-tuning training process may be applied to the pre-trained model 801 using a relatively small subject-specific dataset to obtain a subject-specific model 802 which is fine-tuned for accuracy with respect to a specific subject. In embodiments, the subject-specific model 802 may correspond to the model 710 discussed above, but embodiments are not limited thereto.

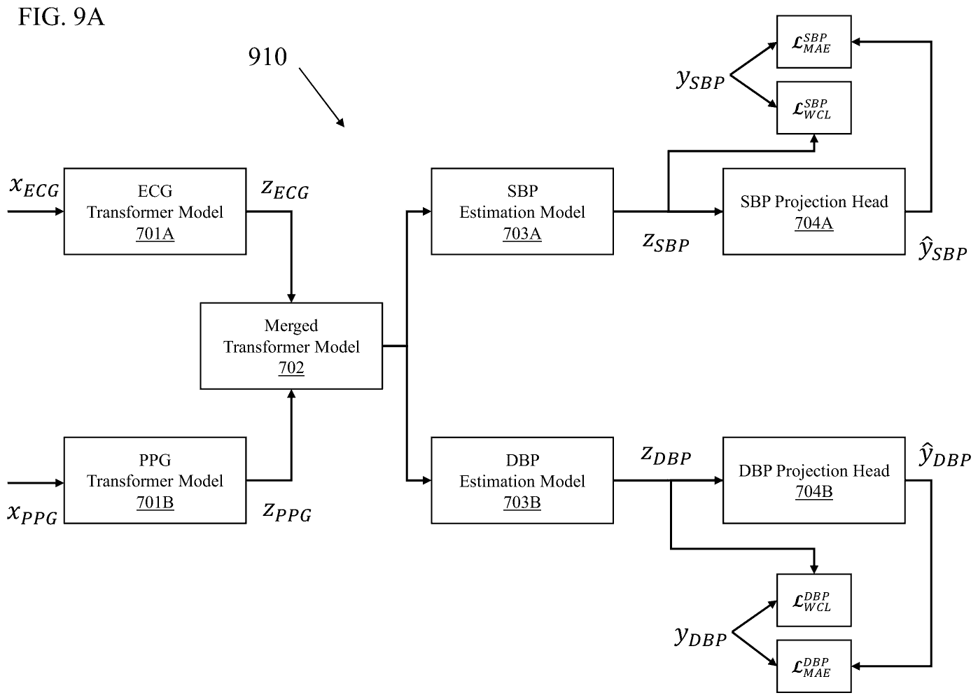

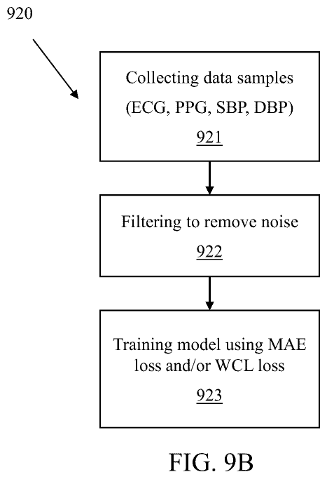

FIG. 9A is a block diagram illustrating an overview of a training process for training a model for performing cuffless BP monitoring, according to embodiments. According to embodiments, after the training process is completed, the trained model 910 may correspond to at least one from among the model 710, the pre-trained model 801, and the subject-specific model 802 discussed above.

As can be seen in FIG. 9A, a model 910 may be trained using one or more of the MAE loss discussed above and the WCL loss discussed above. For example, according to embodiments an SBP MAE loss [symbol] _{MAE}^{SBP} may be determined based on the ground truth SBP values γ_{SBP} and the predicted SBP value \hat{γ}_{SBP}, and an SBP WCL loss [symbol] _{WCL}^{SBP} may be determined based on the ground truth SBP values γ_{SBP} and the SBP embeddings Z_{SBP}. Similarly, a DBP MAE loss [symbol] _{MAE}^{DBP} may be determined based on the ground truth DBP values γ_{DBP} and the predicted DBP value \hat{γ}_{DBP}, and a DBP WCL loss [symbol] _{WCL}^{DBP} may be determined based on the ground truth DBP values γ_{DBP} and the DBP embeddings Z_{DBP}. In embodiments, at least one of the pre-training process and the subject-specific training process may be performed based on any combination of one or more of the SBP MAE loss [symbol] _{MAE}^{SBP}, the DBP MAE loss [symbol] _{MAE}^{DBP}, the SBP WCL loss [symbol] _{WCL}^{SBP}, and the DBP WCL loss [symbol] _{WCL}^{DBP}.

FIG. 9B is a flowchart illustrating an example training process for training a model for performing cuffless BP monitoring, according to embodiments. According to embodiments, process 920 shown in FIG. 9B may be performed by any of the elements discussed above with reference to FIGS. 1-6, 7A, 8, and 9A. In some embodiments, the process 920 shown in FIG. 9B may be referred to as a training process or a training phase.

As shown in FIG. 9B, at operation 921, the process 920 may include collecting data samples. For example, in some embodiments, the data samples may include one or more of a large and diverse multimodal dataset which may be used for pre-training, and a smaller subject-specific dataset which may be used for fine-tuning training. In embodiments, each of the data samples may include a tuple including one or more of ECG signals x_{ECG} for a predetermined time period, PPG signals x_{PPG} for the predetermined time period, a ground truth SBP value γ_{SBP} corresponding to the predetermined time period, and a ground-truth DBP value γ_{DBP} corresponding to the predetermined time period.

As further shown in FIG. 9B, at operation 922, the process 920 may include, after the data samples are collected, performing alignment on the samples and filtering to remove noise. As discussed above with reference to operation 722 of process 720, in some embodiments the process 920 may not include operation 922. However, if alignment and/or filter are performed at operation 922 when the process 920 is performed on a particular model, it may be beneficial to perform the same or similar alignment and filtering during operation 722 when the process 720 performed using that model.

As further shown in FIG. 9B, at operation 923, the model 910 may be trained. For example, at operation 923, the ECG signals x_{ECG} and PPG signals x_{PPG} from the collected samples may be passed through the model 910 to obtain the SBP embeddings z_{SBP} and the DBP embeddings z_{DBP}. Then, the SBP embeddings z_{SBP} may be passed through the SBP projection head 704A to obtain the predicted SBP values \hat{γ}_{SBP}, and the DBP embeddings z_{DBP} may be passed through the DBP projection head 704B to obtain the predicted DBP values \hat{γ}_{DBP}.

According to embodiments, a training process performed according to the process 920 may include two stages, a pre-training stage and a fine-tuning stage. In embodiments, operations 921 through 923 may be performed in each of the pre-training stage and the fine-tuning stage, but embodiments are not limited thereto. For example, in some embodiments all of the training data may be collected and prepared at operation 921 and operation 922, and then operation 923 may be performed for the pre-training stage, and then performed again for the fine-tuning stage.

The pre-training stage may correspond to the pre-training process discussed above with reference to FIG. 8, and may be used to train the pre-trained model 801. In embodiments, the pre-training stage may be performed based on multi-modal data as a way to overcome the difficulty of collecting subject-specific data. The goal of the pre-training stage may be to learn from generalized data so that this knowledge may be transferred to specific subjects. According to embodiments, the pre-training phase may be performed in various ways. For example, in some embodiments only WCL loss may be used for the pre-training stage, in which case the projection heads may be not trained during the pre-training stage. The WCL loss may be determined based on the SBP embeddings z_{SBP} and the DBP embeddings z_{DBP}, with the similarity metrics being computed based on their respective ground truths. As another example, in some embodiments only the prediction error (e.g., the MAE loss) may be used for pre-training, in which case the projection heads may be also trained. As yet another example, both the WCL loss and the predictive loss may be used for the pre-training stage. For example, in some embodiments the SBP MAE loss [symbol] _{MAE}^{SBP}, the DBP MAE loss [symbol] _{MAE}^{DBP}, the SBP WCL loss [symbol] _{WCL}^{SBP}, and the DBP WCL loss [symbol] _{WCL}^{DBP} may be combined, for example using addition or weighted addition, and the resulting composite loss may be used to perform the pre-training stage. In addition, in some embodiments, some or all of the subject-specific dataset may be combined with the large and diverse dataset to perform the pre-training.

The fine-tuning stage may correspond to the fine-tuning training process discussed above with reference to FIG. 8, and may be used to train the subject-specific model 802. In embodiments, the fine-tuning training process may be performed based on a relatively small subject-specific dataset. For example, the fine-tuning training process may be performed based on a dataset which includes data from only one subject, or a small number of subjects, and a number of samples used to perform the fine-tuning training process may be smaller than a number of samples included in the dataset used to perform the pre-training, and may also be smaller than the subject-specific dataset used in other approaches in which the models are trained solely based on prediction error loss. According to embodiments, any combination of one or more of the prediction error loss (e.g., the MAE loss) and the WCL loss may be used in the fine-tuning stage.

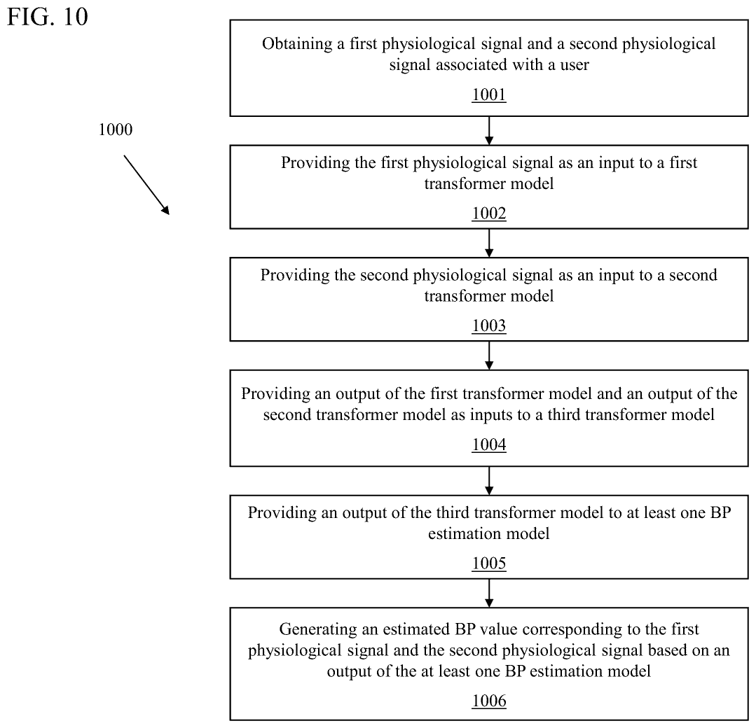

FIG. 10 is a flowchart illustrating an example process for performing cuffless BP monitoring, according to embodiments. According to embodiments, process 1000 shown in FIG. 10 may be performed by any of the elements discussed above with reference to FIGS. 1-6, 7A, 8, and 9A.

As shown in FIG. 10, at operation 1001 the process 1000 may include obtaining a first physiological signal and a second physiological signal associated with a user. In embodiments, the user may correspond to a subject discussed above. In embodiments, the first physiological signal may correspond to the ECG signals x_{ECG} discussed above, and the second physiological signal may correspond to the PPG signals x_{PPG} discussed above.

As further shown in FIG. 10, at operation 1002 the process 1000 may include providing the first physiological signal as an input to a first transformer model. In embodiments, the first transformer model may correspond to the ECG transformer model 711A discussed above.

As further shown in FIG. 10, at operation 1003 the process 1000 may include providing the second physiological signal as an input to a second transformer model. In embodiments, the second transformer model may correspond to the PPG transformer model 711B discussed above.

As further shown in FIG. 10, at operation 1004 the process 1000 may include providing an output of the first transformer model and an output of the second transformer model as inputs to a third transformer model. In embodiments, the third transformer model may correspond to the merged transformer model 712 discussed above.

As further shown in FIG. 10, at operation 1005 the process 1000 may include providing an output of the third transformer model to at least one BP estimation model. In embodiments, the at least one BP estimation model may correspond to at least one of the SBP estimation model 713A discussed above, and the DBP estimation model 713B discussed above.

As further shown in FIG. 10, at operation 1006 the process 1000 may include Generating an estimated BP value corresponding to the first physiological signal and the second physiological signal based on an output of the at least one BP estimation model. In embodiments, the estimated BP value may correspond to at least one from among the predicted SBP value \hat{γ}_{SBP} discussed above and the predicted DBP value \hat{γ}_{DBP} discussed above.

In embodiments, the first transformer model, the second transformer model, the third transformer model, and the at least one BP estimation model may be trained using a pre-training process and a user-specific training process, wherein the pre-training process is performed using a first training dataset corresponding to a plurality of users, and wherein the user-specific training process is performed based on a second training dataset corresponding to the user. In embodiments, the pre-training process may correspond to the pre-training process discussed above with reference to FIG. 8, and the pre-training stage discussed above with reference to FIGS. 9A-9B. In embodiments, the user-specific training process may correspond to the fine-tuning training process discussed above with reference to FIG. 8, and the fine-tuning stage discussed above with reference to FIGS. 9A-9B

In embodiments, at least one of the pre-training process and the user-specific training process is performed using a weighted contrastive loss function comprising a similarity metric. In embodiments, the weighted contrastive loss function and the similarity metric may correspond to the WCL loss [symbol] _{WCL} and the similarity metric Sᵢⱼ discussed above with reference to Equation 2 and Equation 3.

In embodiments, the similarity metric may indicate a similarity between a first ground truth BP value corresponding to a first training sample and a second ground truth BP value corresponding to a second training sample.

In embodiments, the similarity metric may be used to cluster training samples included in at least one of the first training dataset and the second training dataset in an embedding space.

In embodiments, the process 1000 may further include providing the output of the third transformer model to the SBP estimation model; generating an estimated SBP value based on an output of the SBP estimation model; providing the output of the third transformer model to the DBP estimation model; and generating an estimated DBP value based on an output of the DBP estimation model, and the estimated BP value may include the estimated SBP value and the estimated DBP value.

According to one or more embodiments of the disclosure, a method may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in a machine-readable storage medium, such as memory of the manufacturer's server, a server of the application store, or a relay server.

According to one or more embodiments, each component (e.g., a module or a program) of the above-described components may include a single entity or multiple entities. According to one or more embodiments, one or more of the above-described components may be omitted, or one or more other components may be added. Alternatively or additionally, a plurality of components (e.g., modules or programs) may be integrated into a single component. In such a case, according to one or more embodiments, the integrated component may still perform one or more functions of each of the plurality of components in the same or similar manner as they are performed by a corresponding one of the plurality of components before the integration. According to one or more embodiments, operations performed by the module, the program, or another component may be carried out sequentially, in parallel, repeatedly, or heuristically, or one or more of the operations may be executed in a different order or omitted, or one or more other operations may be added.

According to one or more embodiments, in a non-volatile storage medium storing instructions, the instructions may be configured to, when executed by at least one processor, cause the at least one processor to perform at least one operation. The at least one operation may include displaying an application screen of a running application (e.g., the application 146) on a display (e.g., the display 160), identifying a data input field included in the application screen, identifying a data type corresponding to the data input field, displaying at least one external electronic device, around the electronic device, capable of providing data corresponding to the identified data type, receiving data corresponding to the identified data type from an external electronic device selected from among the at least one external electronic device through a communication circuit, and entering the received data into the data input field.

The embodiments of the disclosure described in the specification and the drawings are only presented as specific examples to easily explain the technical content according to the embodiments of the disclosure and help understanding of the embodiments of the disclosure, not intended to limit the scope of the embodiments of the disclosure. Therefore, the scope of one or more embodiments of the disclosure should be construed as encompassing all changes or modifications derived from the technical spirit of one or more embodiments of the disclosure in addition to the embodiments disclosed herein.

What is claimed is:

1. A method for performing cuffless blood pressure (BP) measurement, the method comprising:
obtaining a first physiological signal and a second physiological signal associated with a user;
providing the first physiological signal as an input to a first transformer model;
providing the second physiological signal as an input to a second transformer model;
providing an output of the first transformer model and an output of the second transformer model as inputs to a third transformer model;
providing an output of the third transformer model to at least one BP estimation model; and
generating an estimated BP value corresponding to the first physiological signal and the second physiological signal based on an output of the at least one BP estimation model.

2. The method of claim 1, wherein the first physiological signal comprises an electrocardiogram (ECG) signal associated with the user, and
wherein the second physiological signal comprises a photoplethysmogram (PPG) signal associated with the user.

3. The method of claim 1, wherein the first transformer model, the second transformer model, the third transformer model, and the at least one BP estimation model are trained using a pre-training process and a user-specific training process,
wherein the pre-training process is performed using a first training dataset corresponding to a plurality of users, and
wherein the user-specific training process is performed based on a second training dataset corresponding to the user.

4. The method of claim 3, wherein at least one of the pre-training process and the user-specific training process is performed using a weighted contrastive loss function comprising a similarity metric.

5. The method of claim 4, wherein the similarity metric indicates a similarity between a first ground truth BP value corresponding to a first training sample and a second ground truth BP value corresponding to a second training sample.

6. The method of claim 4, wherein the similarity metric is used to cluster training samples included in at least one of the first training dataset and the second training dataset in an embedding space.

7. The method of claim 1, wherein the at least one BP estimation model comprises a systolic BP (SBP) estimation model and a diastolic BP (DBP) estimation model.

8. The method of claim 7, further comprising:
providing the output of the third transformer model to the SBP estimation model;
generating an estimated SBP value based on an output of the SBP estimation model;
providing the output of the third transformer model to the DBP estimation model; and
generating an estimated DBP value based on an output of the DBP estimation model,
wherein the estimated BP value comprises the estimated SBP value and the estimated DBP value.

9. An electronic device for performing cuffless blood pressure (BP) measurement, the electronic device comprising:
a first sensor configured to obtain a first physiological signal from a user;
a second sensor configured to obtain a second physiological signal from the user; and
at least one processor configured to:
provide the first physiological signal as an input to a first transformer model;
provide the second physiological signal as an input to a second transformer model;
provide an output of the first transformer model and an output of the second transformer model as inputs to a third transformer model;
provide an output of the third transformer model to at least one BP estimation model; and
generate an estimated BP value corresponding to the first physiological signal and the second physiological signal based on an output of the at least one BP estimation model.

10. The electronic device of claim 9, wherein the first physiological signal comprises an electrocardiogram (ECG) signal associated with the user, and
wherein the second physiological signal comprises a photoplethysmogram (PPG) signal associated with the user.

11. The electronic device of claim 9, wherein the first transformer model, the second transformer model, the third transformer model, and the at least one BP estimation model are trained using a pre-training process and a user-specific training process,
wherein the pre-training process is performed using a first training dataset corresponding to a plurality of users, and
wherein the user-specific training process is performed based on a second training dataset corresponding to the user.

12. The electronic device of claim 11, wherein at least one of the pre-training process and the user-specific training process is performed using a weighted contrastive loss function comprising a similarity metric.

13. The electronic device of claim 12, wherein the similarity metric indicates a similarity between a first ground truth BP value corresponding to a first training sample and a second ground truth BP value corresponding to a second training sample.

14. The electronic device of claim 12, wherein the similarity metric is used to cluster training samples included in at least one of the first training dataset and the second training dataset in an embedding space.

15. The electronic device of claim 9, wherein the at least one BP estimation model comprises a systolic BP (SBP) estimation model and a diastolic BP (DBP) estimation model.

16. The electronic device of claim 15, wherein the at least one processor is further configured to:
provide the output of the third transformer model to the SBP estimation model;
generate an estimated SBP value based on an output of the SBP estimation model;
provide the output of the third transformer model to the DBP estimation model; and
generate an estimated DBP value based on an output of the DBP estimation model,
wherein the estimated BP value comprises the estimated SBP value and the estimated DBP value.

17. A non-transitory computer-readable medium storing instructions which, when executed by at least one processor of a device for performing cuffless blood pressure (BP) measurement, cause the device to:
obtain a first physiological signal and a second physiological signal associated with a user;
provide the first physiological signal as an input to a first transformer model;
provide the second physiological signal as an input to a second transformer model;
provide an output of the first transformer model and an output of the second transformer model as inputs to a third transformer model;
provide an output of the third transformer model to at least one BP estimation model; and
generate an estimated BP value corresponding to the first physiological signal and the second physiological signal based on an output of the at least one BP estimation model.

18. The non-transitory computer-readable medium of claim 17, wherein the first physiological signal comprises an electrocardiogram (ECG) signal associated with the user, and
wherein the second physiological signal comprises a photoplethysmogram (PPG) signal associated with the user.

19. The non-transitory computer-readable medium of claim 17, wherein the first transformer model, the second transformer model, the third transformer model, and the at least one BP estimation model are trained using a pre-training process and a user-specific training process,
wherein the pre-training process is performed using a first training dataset corresponding to a plurality of users, and
wherein the user-specific training process is performed based on a second training dataset corresponding to the user.

20. The non-transitory computer-readable medium of claim 19, wherein at least one of the pre-training process and the user-specific training process is performed using a weighted contrastive loss function comprising a similarity metric.
